(19) United States
(12) Patent Application Publication
(10) Pub. No.: US 2009/0221573 A1
(43) Pub. Date: Sep. 3, 2009

(54) Use of Activators of Soluble Guanylate Cyclase for Promoting Wound Healing

(75) Inventors: Thomas Krahn, Hagen (DE); Johannes-Peter Stasch, Solingen (DE); Gerrit Weimann, Köln (DE); Wolfgang Thielemann, Wuppertal (DE); Beatrix Stelte-Ludwig, Wülfrath (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/988,351

(22) PCT Filed: Jul. 6, 2006
(86) PCT No.: PCT/EP2006/006598

§ 371 Date: May 12, 2009

(30) Foreign Application Priority Data
Jul. 6, 2005 (DE) 102005031575.5

(51) Int. Cl. C07D 413/12; C07D 471/04; A61K 31/5377; C07D 401/14; A61K 31/195; C07D 413/14; A61K 31/495; A61K 31/44; C07C 229/34

(52) U.S. Cl. 514/234.2; 514/231.5; 514/236.8; 514/256; 514/567; 544/122; 544/137; 544/146; 544/328; 562/443

(57) ABSTRACT

The present invention relates to a method for promoting wound healing by administering one or more compounds identified in the claims, and to pharmaceutical compositions containing such compounds.

The present invention relates to the use of substances for manufacturing a pharmaceutical product/medicament for promoting wound healing.

The process of wound healing and the regulating of healing of injured tissue generally involves the recruitment of inflammatory cells, followed by fibroblasts, which accumulate in the region of the wound. Collagen and other connective tissue constituents are deposited and transposed in order to restore the original connective tissue (e.g. in skin, muscle, ligaments and tendons). It is therefore of great clinical value to find medicaments which promote this process following surgical interventions, injuries or where wound healing is impaired. Wound healing is impaired, i.e. delayed or inhibited, e.g. in patients with systemic disorders such as diabetes, renal or hepatic damage, peripheral vascular disorders and in patients who take medicaments which impair wound healing, e.g. corticosteroids, immunosuppressants and substances for inhibiting vessel growth.

Any interruption of the anatomical or physiological function of a body tissue is defined as a wound. This means that not just a cut which injures the skin is a wound. If an internal organ no longer functions correctly, this is also a wound. These physiological wounds are often regarded more as an injury. A distinction is therefore also made between open and closed wounds. Open wounds include for example incision, puncture, laceration, tear, abrasion, bite and gunshot wounds, and excoriations and traumatic amputations. Closed wounds include for example contusions, bruises, distortions, burns, frostbites, chemical wounds by acids or alkalis, actinic wounds and necroses (e.g. tissue damage resulting from deficient blood flow, e.g. myocardial and cerebral infarctions).

The aim of wound healing is complete regeneration, i.e. restoration of the normal condition of the tissue. However, the regeneration processes are not always able to achieve this condition. Frequently, therefore, only incomplete regeneration takes place, e.g. if deeper-lying layers of skin (e.g. dermis) are affected. Also affected are tissues which are scarcely capable of renewal (cardiac and nerve tissues). In these cases, the lost tissue is usually filled out by replacement tissue (connective tissue) which is of low functional quality and has a poorer blood supply and is less elastic and functional than the original tissue. This is referred to as scar or scar tissue. In order to recover maximum functionality, therefore, it may also be an aim to promote the regeneration of functional tissue (e.g. muscle cells) by comparison with connective tissue (fibroblasts).

It is known that mechanisms (e.g. NO-releasing substances) which lead to an increase in the intracellular messenger cGMP can also contribute to improving wound healing (WO 96/08966-A1, Murrell).

Activation (caused by agonists) of soluble guanylate cyclase leads to an increase in the intracellular messenger cGMP. It has surprisingly now been found that the compounds I-VI listed below, which are activators of soluble guanylate cyclase according to the invention, are suitable for manufacturing pharmaceutical substances/medicaments for promoting wound healing, especially in humans.

Compound (I) corresponds to the following formula:

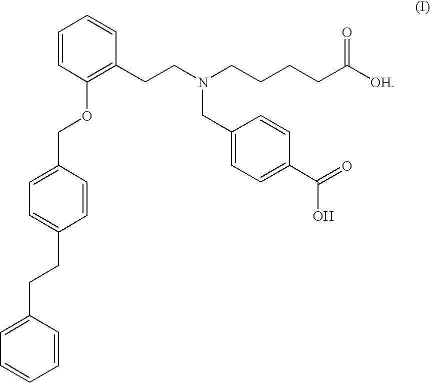

Compound (I), its preparation and use as medicament have been disclosed in WO 01/19780.

Compound (II) corresponds to the following formula:

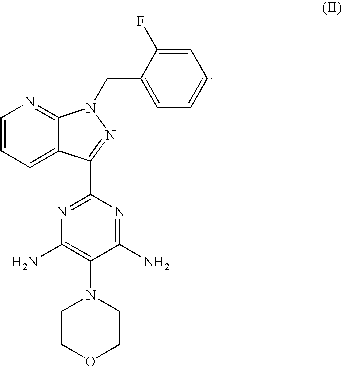

Compound (II), its preparation and use as medicament have been disclosed in WO 00/06569.

Compound (III) corresponds to the following formula:

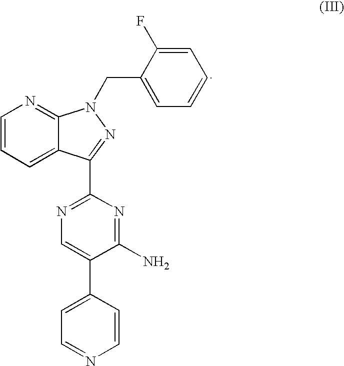

Compound (III), its preparation and use as medicament have been disclosed in WO 00/06569 and WO 02/42301.

Compound (IV) corresponds to the following formula:

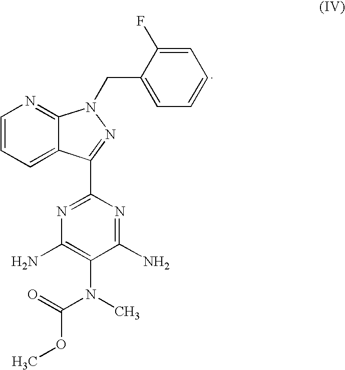

Compound (IV), its preparation and use as medicament have been disclosed in WO 00/06569 and WO 03/095451.

Compound (IVa) corresponds to the following formula:

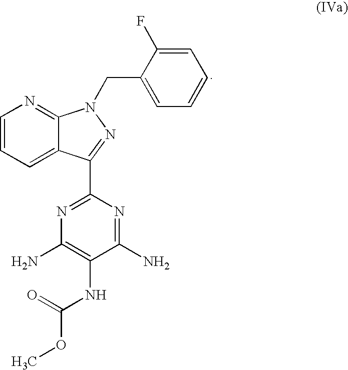

Compound (IVa), its preparation and use as medicament have been disclosed in WO 00/06569 and WO 03/095451.

Compound (V) corresponds to the following formula:

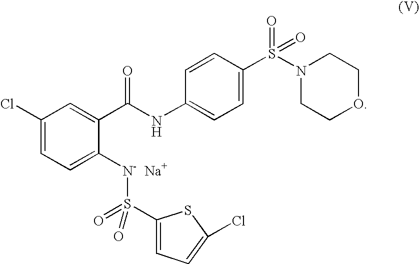

Compound (VI) corresponds to the following formula:

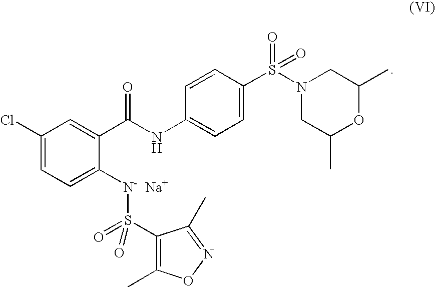

Compounds (V) and (VI), their preparation and use as medicaments have been disclosed in WO00/02851.

The present invention relates to the use of compounds of the formulae (I-VI) and their salts, hydrates, and hydrates of the salts for manufacturing a medicament for promoting wound healing.

The present invention further relates to the pharmaceutical composition which comprises at least one compound of the formulae (I-VI) with a pharmaceutically acceptable carrier, in each case for each of the therapeutic effects discussed previously. The composition can be given alone or in combination with at least one further substance, e.g. a stabilizing substance.

The compounds of the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or otic route or as implant or stent.

The compounds of the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilisates or sterile powders.

Examples suitable for other administration routes are medicinal forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking tastes and/or odors.

The present invention further relates to medicaments which comprise at least one compound of the formulae (I-VI) of the invention, normally together with one or more inert, non-toxic, pharmaceutically suitable excipients, and to the use thereof for the aforementioned purposes.

It has generally proved advantageous to administer amounts of about 0.01 to 5000 mg/kg, preferably about 0.5 to 1000 mg/kg of body weight per day to achieve effective results.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The present invention preferably relates to the topical use of a pharmaceutical formulation.

The present invention further relates to suspensions and ointments for topical use which comprise at least one of the compounds of the formulae (I-VI). Further topical preparations of the invention include solutions, sprays, lotions, gels, creams, powders, powder sprays, pastes, emulsions, foams and sticks which comprise at least one active ingredient of the formula (I-VI), where appropriate also a plurality of active ingredients.

Topical application of an active ingredient of the formula (I-VI) also takes place in the form of plasters, film dressing sprays, occlusive dressings, compresses and controlled delivery systems. The active ingredients may be present in these preparations in dissolved or suspended form.

Ointments comprise hydrocarbon gels, lipogels, absorption bases, W/O ointment bases, mixed emulsions or polyethylene glycols as base.

Creams comprise O/W bases.

Pastes comprise large amounts of powdered ingredients such as, for example, zinc oxide, talc, starch or titanium dioxide, besides an ointment or cream base.

Gels comprise solvents such as water, ethanol, isopropanol or propylene glycol and are produced with use of gel formers such as cellulose ethers, alginates, polyacrylates, bentonite, gelatin, tragacanth, polyvinylpyrrolidone or polyvinyl alcohol. The use of lipophilic gel bases or of micro-emulsions is also possible.

Dusting powders comprise powdered additives such as starch, stearates, silicon dioxide, clay, magnesium carbonate, talc, cellulose, zinc oxide and especially lactose.

It is possible to add stabilizers, antioxidants, preservatives, humectants, superfatting agents, solvents or excipients to improve penetration and activity to all the preparations.

Examples of penetration improvers are propylene glycol, polyethylene glycol, dimethyl sulfoxide, decyl methyl sulfoxide, azone, N-methylpyrrolidone, diethyltoluamide, ethanol, isopropyl myristate, isopropyl palmitate, oleic acid and its esters, medium chain triglycerides, dimethylisosorbitol, 2-octyldodecanol, branched-chain fatty acid esters, benzyl alcohol, urea, salicylates and surfactants.

A further pharmaceutical formulation suitable for topical use which can be used is a carrier system based on liposomes which is known from the prior art of pharmaceutical technology. This system ought in particular to improve the penetration of lipids into the epidermis. Such liposome dispersions with various inclusion compounds and phospholipids as bilayer membrane formers or so-called empty liposomes without inclusion compounds have been described in numerous publications and already tested clinically. Production takes place for example by treating an aqueous phospholipids dispersion with ultrasound, dispersing phospholipids with surfactants in aqueous phase, dissolving phospholipids in organic solvents, removing the solvent by lyophilization and dispersing the residue in aqueous phase, infusion methods or reverse phase evaporation. Preferred pharmaceutical formulations for topical use are microdisperse systems based on lipid mixtures.

The document WO 96/37192 discloses pharmaceutical or cosmetic compositions which comprise, in combination with a sphingolipid or glycolipid (ceramide), the following: a partial fatty acid ester of polyoxyethylene sorbitan, a phospholipid, a triglyceride and a therapeutic active ingredient, in water (and possibly alkanol) as carrier liquid. EP 0852941 discloses compositions in which the active ingredients can be solubilized in the form of a dispersion of nanoparticles (nanodispersion) by a similar excipient mixture of the type of partial fatty acid esters of polyoxyethylene sorbitan, phospholipid and ethanol, also water-insoluble, liquid to highly viscous, oily or at any rate oil-soluble active ingredients, e.g. lipid-soluble vitamins, therapeutic oils or photoprotective substances. The pharmaceutical formulations, described in this disclosure, comprising compounds of the formulae (I-VI) are regarded as preferred subject matter of the present invention.

The formulations can moreover comprise, appropriate for the intervention, active substance between 0.1 and 99% active ingredient, in a suitable manner 25-95% in the case of tablets and capsules and 1-50% in the case of liquid formulations, i.e. the active ingredient should be present in amounts sufficient to achieve the stated dose range.

The present invention further relates to the use of a combination of one or more of the compounds (I-VI) of the invention with one or more other substances. Examples of suitable combination partners are substances which promote vessel growth. Mention is made by way of example and preferably in this connection of cGMP-increasing substances, adenosine agonists and growth factors.

The present invention further relates to the use of a combination of one or more of the compounds (I-VI) of the invention with one or more substances selected from the group of medicaments for promoting blood flow, for pain relief or else of antibiotics.

### EXPERIMENTAL PART

### 1. Influencing the Migration of Smooth Muscle Cells In Vitro by Administering an Activator of Soluble Guanylate Cyclase

Smooth muscle cells of the human coronary artery are seeded in a 6-well plate (1.5×10⁵ cells/well) (Clonetics) and cultured in SmGM2 medium (Clonetics) for 48 h. Before the plates are used, the wells are coated with vitronectin (50 ng/cm²; Gibco BRL). Half of the confluent cell monolayer is removed with the aid of a cell scraper (wounding). In the cell-free region, the vitronectin coating is at least 50% retained in this case. For the test, unless mentioned otherwise, the culture medium is replaced by MCDB-131/0.2% BSA (Gibco/BRL) medium.

The test substances are added in various concentrations, and the migration distance in the cell-free region is followed with the aid of a microscope over 24 h and 48 h. Each measurement point represents the average of four measured regions. PDGF, as potent chemotactic factor for SMC, is used as positive control (1 nM, 10 nM, R&D systems). The RGD tripeptide (Bachem) is employed to inhibit the integrin-induced migration. To determine the antagonistic activity of the test substances, the cells are stimulated in the presence of the substance with 1 nM or 10 nM PDGF, and the migration distances are determined in relation to the PDGF-induced migration distances.

Representative results from this test are shown in the following table:

TABLE
Inhibition of the horizontal migration of smooth muscle cells
of the human coronary artery (incubation time 48 h)

| Compound | IC₅₀ [nM] |
|---|---|
| I | 5 |
| IV | 10 |
| IVa | 30 |

Compounds I-VI show that activators of soluble guanylate cyclase are suitable for promoting wound healing.

### 2. Formulation of the Active Ingredients

### Opthalmological Topical Use

To form a 2% by weight suspension, the active ingredients are mixed with Liquifilm® eye drops (Allergan, Ettlingen, Germany) (1 ml containing polyvinyl alcohol 14 mg, and as excipient chlorobutanol×H₂O 5.25 mg, sodium chloride) and sonified in a glass tube in an ultrasonic bath at 15° C. for 15 min.

### Dermatological Topical Use

### 1) Alcoholic Suspension

To form a 2% by weight suspension, the active ingredients are mixed with 30% by weight isopropyl myristate/70% by weight of ethanol and sonified in a glass tube in an ultrasonic bath at 15° C. for 15 min.

2) Spreadable Preparations

To produce the ointment preparation, wool wax alcohol ointment complying with DAB 9 is employed. The active ingredient is suspended in a molten mixture of 0.5 part by weight of cetylstearyl alcohol (DAB 9 quality), 6 parts by weight of wool wax alcohol (DAB 9 quality) and 93.5 parts by weight of white petrolatum (DAB 9 quality). The mixture is stirred while cooling to room temperature (about 21° C.).

To produce a so-called “PEG isogel” preparation, the active ingredient is suspended or dissolved in the melt of a mixture of low molecular weight polyethylene glycol (PEG) and higher molecular weight PEG. After the active ingredient has been suspended or dissolved in the melt, the preparation is stirred while cooling to room temperature.

Lower molecular weight PEG is liquid at room temperature (about 21° C.), while higher molecular weight PEG is solid (can be cut, wax-like) at room temperature.

The mixing ratios of the two PEG types depends on the required viscosity of the preparation and on the average molecular weights of the PEG types employed.

Examples which can be used are preparations of 6.5 parts by weight of PEG 400 and 1 part by weight of PEG 4000 or 7 parts by weight of PEG 400 and 2 parts by weight of PEG 6000. Mixing ranges vary from 1:1 to 10:1 (in each case parts by weight of low molecular weight PEG to higher molecular weight PEG). The range from 2:1 to 8:1 is preferred, and the range from 3:1 to 7:1 is particularly preferred.

A further gel preparation employed is a polyacrylate gel (so-called “carbogel”) which consists of 1 part by weight of Carbopol 974 P NF (manufacturer BF Goodrich, USA), 5 parts by weight of isopropyl alcohol, 5 parts by weight of sodium hydroxide solution (5% by weight) and 89 parts by weight of water. The preparation is produced by rubbing the carbopol with the isopropanol, adding the active ingredient and dispersing the mixture in water. The sodium hydroxide solution is added stepwise while stirring. A gel is formed.

1. A method for promoting wound healing, comprising administering an effective amount of a compound selected from the group consisting of compounds having the formulae (I-IV) 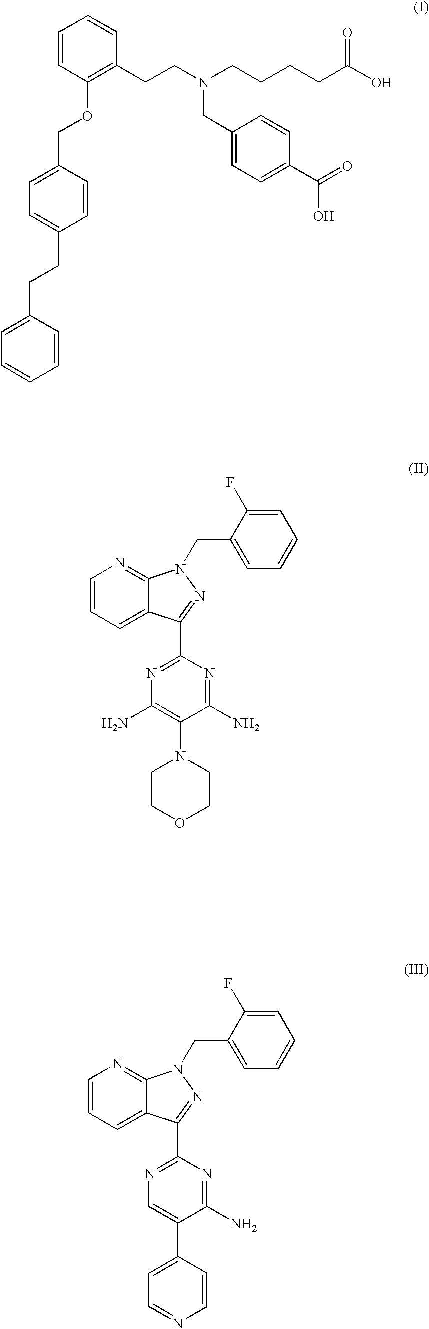 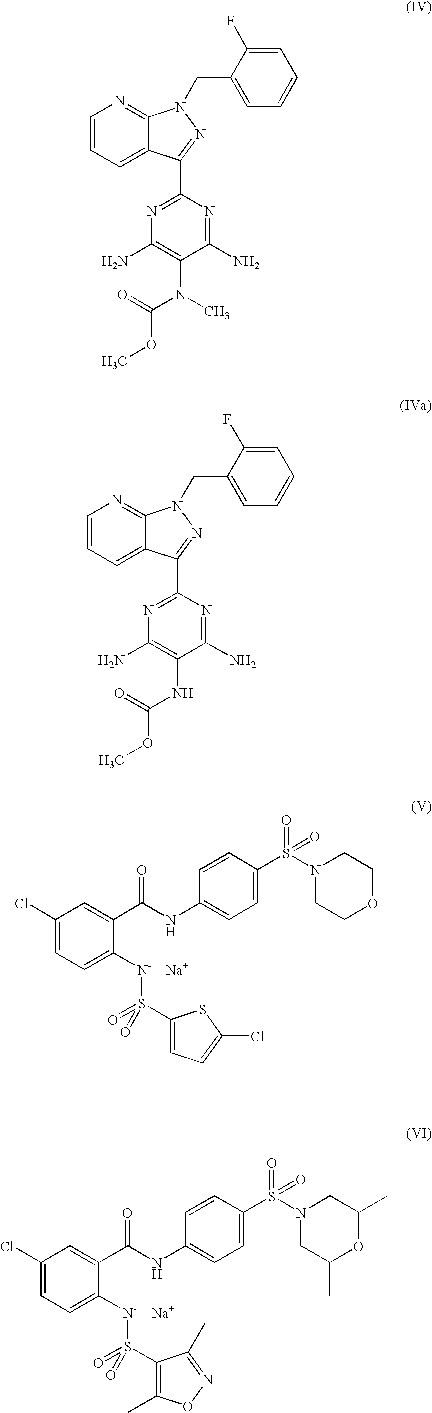
or a salt, hydrate, or hydrate of a salt thereof.

2. The method as claimed in claim 1, in which the compound is administered in a topical dosage form.

3. The method as claimed in claim 1, in which the compound is administered subcutaneously or intramuscularly.

4. The method as claimed in claim 1, in which the compound is used preventively.

5. The method as claimed in claim 1, in which the compound is administered for treating wounds in diabetics.

6. A pharmaceutical composition for promoting wound healing, which comprises at least one compound as claimed in claim 1.

7. The pharmaceutical composition as claimed in claim 6, which additionally comprises a compound for promoting blood flow, for pain relief, or which is an antibiotic.
